(19) United States
(12) Patent Application Publication
(10) Pub. No.: US 2016/0078787 A1
(43) Pub. Date: Mar. 17, 2016

(54) Medical Band Assembly With Detachable Flag Label

(71) Applicants: Kim Canchola, Canyon Country, CA (US); Ronald G. Ennis, Los Angeles, CA (US); Shane Taghavi, Valencia, CA (US)

(72) Inventors: Kim Canchola, Canyon Country, CA (US); Ronald G. Ennis, Los Angeles, CA (US); Shane Taghavi, Valencia, CA (US)

(21) Appl. No.: 14/486,025

(22) Filed: Sep. 15, 2014

(51) Int. Cl. G09F 3/00; B32B 37/14; B32B 38/10; A61B 19/00; B01L 3/00

(52) U.S. Cl. CPC G09F 3/005; A61B 19/44; B01L 3/5453; B32B 38/10; B32B 37/142

(57) ABSTRACT

A medical band assembly for identifying a patient and items and/or samples related to the patient may have an elongated body that may be formed into a loop. The medical band assembly supports a flag label on one of its surfaces. The flag label has a pair of flag sections joined together by an intermediate support bridge. The flag label is separable from the medical band and attachable to an object to be labeled by wrapping the flag label around the object, such that the next extends around the object, and securing the flag sections together on the opposing side.

### CROSS-REFERENCE TO RELATED APPLICATION

Not applicable.

### STATEMENT OF FEDERALLY SPONSORED RESEARCH OR DEVELOPMENT

Not applicable.

### BACKGROUND

This application relates to assemblies for medical bands, such as wristbands, and to labels that are separable from these assemblies for attachment to, for example, blood vials.

During a visit to a hospital, a patient is conventionally provided a medical identification band. This band is affixed to a limb of the patient, typically around a wrist on an arm, usually in a way that precludes easy non-destructive removal of the band from the individual. The band may provide identifying information about the patient such as the patient's name, date of birth, admission date, patient identification number, allergy information and so on. Such bands also may include machine-readable representations such as barcodes or contain a machine-readable chip such as an RFID chip. When a medical professional interacts with this patient or administers medical treatment to the patient, the band may be scanned or read to confirm that the patient is the correct patient to receive the medical treatment and further can be used to record that the treatment was administered.

As one example, if a nurse is administering a medication, then the nurse may scan the medication and then the band of the patient to confirm that medication is being administered and to further confirm that the specific patient is the correct patient to be receiving the medication.

As another example, when a fluid sample (for example, blood) is being taken from the patient, the band may be scanned and then a label printed that may be affixed to a container to associate the patient with the sample. However these labels may be large, effectively blocking view of type of fluid contained within the vial, and make it harder for a nurse or technician to determine what is in the vial through visual inspection.

### SUMMARY

Disclosed herein is a medical band assembly including a medical band with a detachable flag label. The medical band has an elongated body with opposing surfaces bounded at their edges by a periphery. This medical band extends from a main body to a strap. The strap is attachable to the main body to form a loop from the medical band. The flag label is initially supported on one of the opposing surfaces of the main body of the medical band and is separable from the medical band for attachment to an object to be labeled (such as, for example, a vial). The flag label includes a pair of flag sections joined to one another by an intermediate support bridge. When the flag label is separated from the medical band, the flag sections are foldable onto one another to join a contacted pair of their surfaces together such that the intermediate support bridge is wrappable around the object to be labeled by the flag label.

It is contemplated that in many forms of use of the flag label, the intermediate support bridge may wrap around the circumference of the object to be labeled. This is because often the object will be cylindrical in shape, such as if the object is a test tube.

In some forms of the medical band, a transparent laminate shield may be attached to the main body of the medical band such that the transparent laminate shield may be liftable from the main body to separate at least a portion of the transparent laminate shield from the main body. The flag label may be disposed between the medical band and the transparent laminate shield and, when the laminate shield is lifted, the flag label may be detached from the main body of the medical band. In some forms, the transparent laminate shield might include an adhesive backing as well as a removable release liner, such that the laminate shield may be used to cover the main body to prevent the smearing of printing or writing on the flag label or on another label attached to the main body in this region.

In terms of some dimensions for the flag label, the flag section may have a full lateral length between 4.0 and 6.0 inches (from one end of the flag label to the other end of the flag label) and the intermediate support bridge have a lateral length of 1.6 inches and a width between 0.2 and 1.0 inches. In some forms of the medical band assembly, the assembly may further comprise a detachable tail (detachable from the distal end of the strap) having a plurality of tail labels supported thereon. The medical band itself may have one or more main labels supported thereon.

It is contemplated that the flag label, the plurality of the tail labels, and the main label(s) may include a computer readable representation of data (for example, barcodes) and the computer readable representation of data may be the same for the flag label, the plurality of tail labels, and the main label(s).

Additionally or alternatively to the computer readable representation of data, the flag label may further include a human readable representation of data. This data, either human and/or computer readable, may be used to correlate the patient wearing the medical band to any samples that are labeled using flag label or label(s) from the plurality of tail labels.

Also disclosed herein is a method of attaching a flag label to an object. The method includes first detaching the flag label from a medical band assembly of the type described above (in which the medical band assembly includes a medical band having a main body attached to a strap in which the strap is attachable to the main body to form a loop from the medical band and the flag label having first and second flag sections separated by an intermediate support bridge, and can further include an adhesive applied to the back of at least one of the first and second flag sections). With the flag label detached from the medical band assembly, at least a portion of the intermediate support bridge is wrapped around the object and the first flag section is adhered to the second flag section, thereby attaching the flag label around the object.

In some forms of the method, prior to the flag sections being adhered to one another, the first flag section may be aligned with the second flag section.

In some forms of the method, the medical band may further be attached to a wrist of a patient by attaching the strap to the main body to form a loop around the wrist.

Prior to the detaching step, the flag label may be initially releasably adhered to the main body of the medical band. As one example, the medical band itself may serve as a type of release liner for the flag label.

As in the assembly above, the medical band may further include a transparent laminate shield having an adhesive backing protected by a releasable liner. In this instance, the method may further include the step of removing the releasable liner from the transparent laminate shield and adhering the transparent laminate shield to the medical band.

In addition to being on at least one of the flag portions of the flag label, the adhesive may also be supplied or supported on at least a portion of the intermediate support bridge. In this case, the step of adhering may also involve adhering at least a portion of the intermediate support bridge to the object.

In some forms, the intermediate support bridge may be narrower in a width direction than the flag sections such that the intermediate support bridge establishes a neck therebetween.

These and still other advantages of the invention will be apparent from the detailed description and drawings. What follows is merely a description of some preferred embodiments of the present invention. To assess the full scope of the invention the claims should be looked to as these preferred embodiments are not intended to be the only embodiments within the scope of the claims.

### BRIEF DESCRIPTION OF THE DRAWINGS

FIG. 1 is a perspective view of a medical band assembly with a detachable flag label.

FIG. 2 is an exploded perspective view of the medical band assembly of FIG. 1 with the transparent laminate shield flapped back so that the flag label can be detached and with the tail detached from the strap of the medical band.

FIG. 3 is a perspective view of the detached flag label of FIG. 2 partially wrapped around a vial.

FIG. 4 is a perspective view of the detached flag label secured to the vial of FIG. 3.

FIG. 5 is a perspective view of the medical band secured to the wrist of a person or patient.

FIG. 6 is a front view of a second alternative embodiment of the detachable flag label in which the label is generally rectangular.

FIG. 7 is a front view of a third alternative embodiment of the detachable flag label in which the intermediate support bridge connecting the flag portions is located on the opposite side of the label from the U-shaped flag label of FIGS. 1-4.

FIG. 8 is a front view of a fourth alternative embodiment of the detachable flag label in which the intermediate support bridge connecting the flag portions is centrally disposed so that the unattached flag label has a barbell shape.

FIG. 9 is a front view of an alternative embodiment of a medical band with a detachable flag label in which the main body section, the strap section, and the tail are all the same width over the length of the medical band assembly (with the exception of the fastening structure on the end of the medical band).

### DETAILED DESCRIPTION

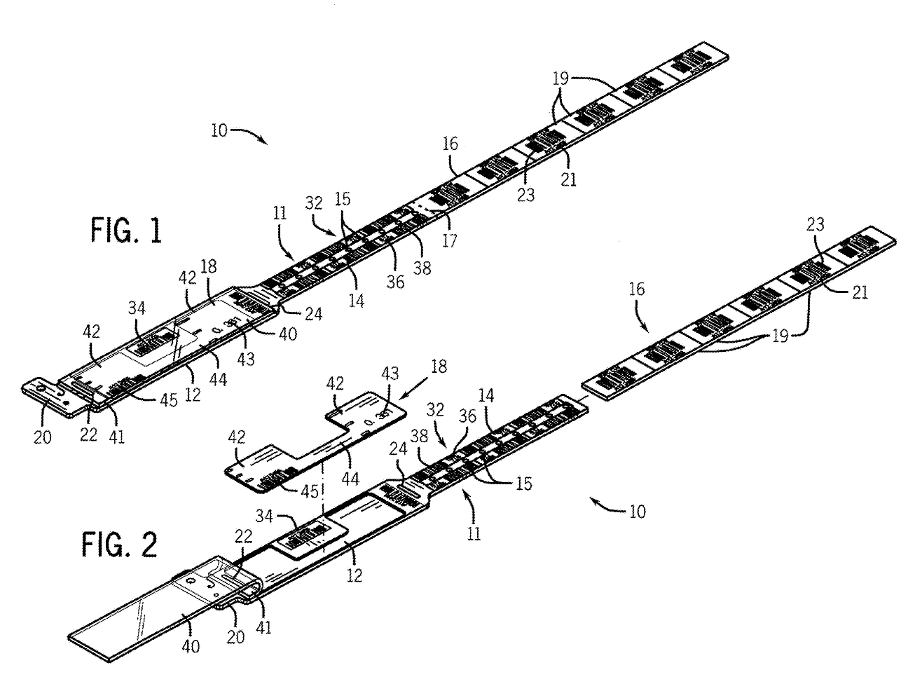

Referring first to FIGS. 1 and 2, a medical band assembly 10 includes a medical band 11 extending from a main body 12 to a strap 14 (which in the form illustrated in FIGS. 1 and 2 are joined at a tapering neck). A plurality of fastener holes 15 extend through the thickness of the strap 14 between the front and back sides. On the distal end of the strap 12 of the medical band 11, there is a separable and detachable tail 16 which is connected to the distal end of the strap 12 at a point of weakness or perforation 17. On the upper surface of the main body 12, a flag label 18 is initially positioned and supported as illustrated in FIG. 1. However, as illustrated in FIG. 2, this flag label 18 is separable and detachable from the main body 12 of the medical band 11 by, for example, peeling the flag label 18 from the main body 12 to which the flag label 18 is releasably adhered. On the distal end of the main body 12 opposite the strap 14, there is a clasp 20 which typically has a fastener in the form of a snap button having a female portion and a male portion. The clasp 20 is foldable inward onto itself to securely capture a portion of the strap 14 between the male and female portions of the fastener to form a loop by locking the portions together through one of the fastener holes 15.

Given that the medical band 11 portion functions as a wristband, the medical band assembly 10 is generally planar in form and has a longer end-to-end length than side-to-side width. The thickness may vary slightly (due to attached shield components, for example), but is otherwise generally uniform over the area of the medical band assembly 10. As illustrated, the main body 12 has a generally rectangular planar shape of a first width and the strap 14 also has a generally rectangular planar shape of a second width that is smaller than the first width. These two sections are integrally connected at a central junction therebetween. Because the main body 12 has a greater width than the strap 14, at this central junction the width “necks down” or tapers between the two sections at the peripheral edge. However, this necking section is certainly not required and, as illustrated in one alternative form in FIG. 9, the main body and the strap sections may be of consistent or uniform width substantially over the length of the assembly (with the exception, for example, of the clasp region).

Additionally, in the form illustrated, slots 22 and 24 extend through the thickness of the main body 12, between the opposing top and the bottom faces. A first slot 22 extends through the main body 12 adjacent to the clasp 20, while the second slot 24 extends through the main body 12 adjacent to the junction between the main body 12 and the strap 14. The slots 22 and 24, as will be described below, can potentially be used to re-attach a medical band 11 that has been attached to a patient and subsequently severed or cut for a first time at the central juncture between the main body 12 and the strap 14.

The strap 14 may have a plurality of identification labels 32 including human readable representation of data (such as, for example, text 36 which is readily viewed and understandable in a sensory manner by a human) and/or computer readable representation of data (such as, for example, barcodes 38, which are more readily interpreted by an optical scanner attached to a computing device). The identification labels 32 may initially be non-destructively attached to the strap 14. For example, the identification labels 32 may have an adhesive backing and the strap 14 may be made of a material that at least partially retains the identification labels 32 on the strap 14, but allows for easy removal of the identification labels 32 that keeps the adhesive on the identification labels 32. The identification labels 32 may be removed from the strap 14 to be applied to paper work, medicine, or sample specimens associated with the patient. Alternatively, the labels 32 on the strap 14 may be permanently affixed to the strap 14 (that is, not be removable) and optionally be covered by a coating or laminate to protect the underlying labels 32.

A main label 34 may be permanently attached to the front side of the main body 12, initially adjacent to the flag label 18, when the flag label 18 is attached to the main body 12. The main label 34 may be a part of the main body 12, or alternatively, may be an adhesive backed label that may be applied to the main body 12, either before or after removal of the flag label 18. The main label 34 may, for example, include the patient's name, date of birth, admission date, patient identification number, and/or allergy information in the form of text 36 and/or a bar code 38 (that is, contain human and/or computer readable representation data). If the main label 34 is permanently attached to the main body 12, then it may be printed directly onto the main body 12, and may be located towards the top vertically and the middle laterally of the main body 12 as illustrated. However, other positioning of the main label 34 is also contemplated based on the geometry of the surrounding flag label 18.

If the main label 34 is not permanently attached to the main body 12, then the main label 34 may have an adhesive applied to its back side with the adhesive being protected by a paper backing. The main label 34 may be preprinted or may have a blank space to allow a nurse to handwrite the patient's name and information. The main label 34 may be attached to the main body 12 by removing the paper backing and pressing the back side of the main label 34 against the front side of the main body 12.

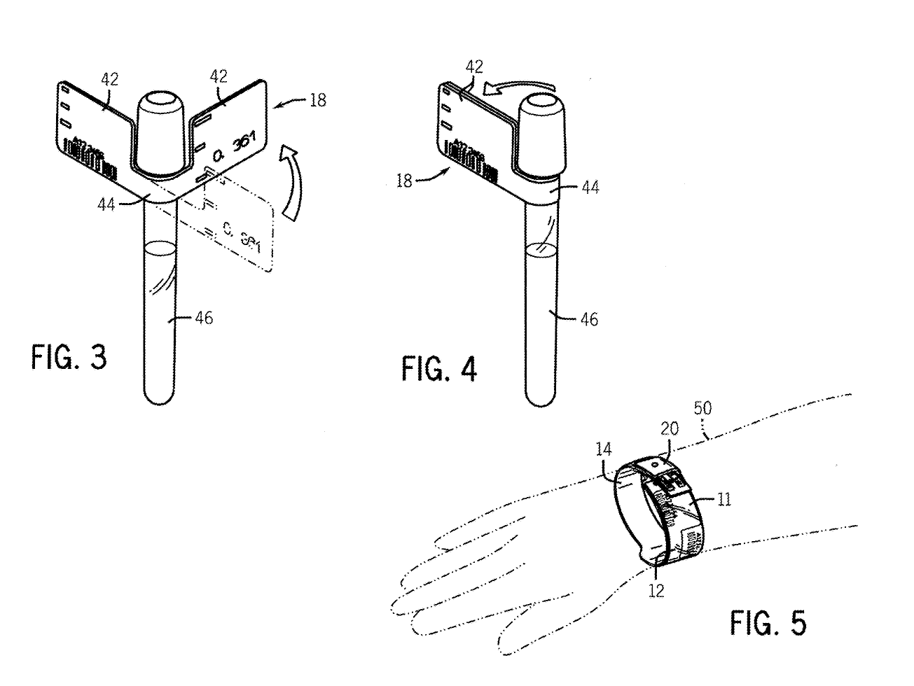

Looking now more closely at the flag label 18, the flag label 18 may come in a variety of shapes and sizes. In one example embodiment, the flag label 18 may generally be in a U shape, having two flag sections 42 connected by an intermediate support bridge 44. The flag sections 42 may be identical, mirror images of one another and may be larger in a width direction than the intermediate support bridge 44, such that the flag label 18 is symmetrical about a line of symmetry extending along the width direction centrally through the intermediate support bridge 44. The corners of the flag label 18 may be rounded to remove any sharp corners, and may also reduce stress around the corners when the flag label 18 is being removed from the medical band assembly 10. The flag label 18 may have an adhesive applied to at least a portion of the back side of the flag label 18 for affixing the flag label 18, for example, to a cylindrical vial 46, or test tube as will be subsequently discussed with reference to FIGS. 3 and 4. The adhesive should at least cover the back of the flag sections 42, such that the flag sections 42 can be attached to one another when they are made to face one another; further, the adhesive may additionally cover the back of the intermediate support bridge 44.

The flag label 18 may have identifying information displayed on the front side of the flag label 18, for example, information may be printed on the flag sections 42, or alternatively, the flag sections 42 may have blank space that may be written on. The flag sections 42 may have the same information on both sections, or alternatively one flag section 42 may display different information than the other flag section 42. In addition to text information 43, the flag section 42 may have a bar code 45; the text information 43 and the barcode 45 may correspond to the text 36 and barcodes 38 used on the main label 34 and the identification labels 32 on the strap 14.

The flag label 18 may initially be removably attached to the main body 12 as differentially depicted between FIGS. 1 and 2. For example, in the attached state in which the flag label 18 is attached to the main body 12, the flag sections 42 may extend substantially between the bottom and top of the main body 12, and may extend from the right and left edges inward toward the centrally-positioned main label 34 which is disposed on one of the lateral peripheral edges. The intermediate support bridge 44 extends vertically along the other lateral peripheral edge of the main body 12. In this configuration, the flag label 18 and the main label 34 may form a generally rectangular shape (with the main label 34 being nested in a central position along one edge of the flag label 18) in order to occupy a substantial top surface area of the main body 12.

In one specific form, the flag label 18 may, for example, have a length of approximately 4.4488 inches and be in a range of 3 to 6 inches long. The flag sections 42 may each have a length of approximately 1.425 inches while the intermediate support bridge 44 may have a length of approximately 1.6 inches and a width in a range of 0.2 to 1.0 inches. However, these dimensions are for purposes of example, and other dimensions may also be used.

A transparent laminate shield 40, or flap, is permanently joined on the distal end to the main body 12, such that the point of connection between the shield 40 and the main body 12 is adjacent to the slot 22 and clasp 20. The transparent laminate shield 40 may have an adhesive backing that faces the upper surface of the main body 12 that is initially protected by a removable release liner (not shown). The transparent shield 40 may be temporarily rotated or folded away from the main body 12 as illustrated in FIG. 2, such that the flag label 18 is accessible for writing or printing on and/or removal from the main body 12 of the medical band 11. Once the release liner of the transparent laminate shield 40 is removed to expose the adhesive backing, the laminate shield 40 can be returned over the main body 12 and pressed and smoothed against the main body 12, such that the adhesive backing comes into contact with the main body 12 to at least semi-permanently attach to the main body 12. Since the shield 40 is transparent, the main label 34 may be seen through the shield 40 as well as the upper surface of the flag label 18, in the event the flag label is not removed from the main body 12. It will be appreciated that instead of an adhesive, the shield 40 and the main body 12 and/or the main label 34 may merely have a contact cling by virtue of their surface properties when contacted, such that the shield 40 may be moved away from, and reattached to, the main body 12.

The tail 16, as mentioned above, may also be detachable from the strap 14, such as through a perforation 17 along the interface between the strap 14 and the tail 16. The tail 16 may contain a plurality of removable identification Labels 19. Each identification label 19 may include text 21, other human-readable representations of data, and/or a barcode 23, or other machine-readable representations of data. The tail 16 may be removed, for example, once all of the identification Labels 19 have been removed. Alternatively, the tail 16 may have adhesive along a portion of its back side, such that the entire tail 16 may be removed and adhered to another surface, such as a sample or documents, and then the individual labels 19 may be removed later.

In order to use the flag label 18, the flag label 18 is removed from the main body 12 of the medical band 11 by rotating open the transparent laminate shield 40, and then pealing the flag label 18 off of the main body 12 as illustrated in FIG. 2. Prior to detachment from the main body 12 of the medical band 11, information may be written or printed on the flag label 18, since the flag label 18 may be easier to write on in the planar attached state.

Now with additional reference to FIGS. 3 and 4, the detached flag label 18 is secured to an object, such as a vial 46 containing a patient sample (for example, blood), by wrapping the intermediate support bridge 44 of the flag label 18 around the vial 46 as best illustrated in FIG. 3. If the intermediate support bridge 44 has adhesive on it, then the intermediate support bridge 44 may be pressed against a portion of the vial 46 or object to temporarily hold the flag label 18 in place, while the flag sections 42 are initially aligned. The flag label 18 may then be secured by aligning the back sides of the flag sections 42 with each other and then contacting and pressing the two sides together, such that the adhesive on the back side of the flag sections 42 stick to one another as illustrated in FIG. 4. Accordingly, in the secured state, the intermediate support bridge 44 encircles the vial 46 (or other object being labeled) and the flag sections 42 are pressed together. Due to the relative thinness of the intermediate support bridge 44 compared to the flag sections 42, more of the vial 46 and the sample volume contained therein can be seen compared to a traditional label which adheres to a larger area on the outside of the vial walls. This allows for the contents of the vial 46 to be more readily ascertained.

Further, it should be appreciated that the flag label may be made more tamper-proof by selecting adhesives and supporting layers that are destroyed upon attempted de-adhesion of the parts of the flag label. For example, the flag label may have a layer/adhesive structure similar to that found in foil adhesive stickers that are used to secure the insides of electronic components for warranty purposes.

In order to apply the medical band 11 to a patient as illustrated in FIG. 5, the medical band assembly 10 is taken and the tail end 16 may be removed. Optionally, the flag label 18 may be written on or removed for attachment to an object as described above. With the medical band 11 properly prepared, the distal end of the strap 14 of the medical band 11 is wrapped around the patient's wrist 50 to form a loop with the distal end of the main body 12 of the medical band 11. The one of the holes 15 in strap 14 is positioned relative to the clasp 20, such that the size of the loop formed is appropriate to the size of the patient's wrist. Once sized, the strap 14 is then secured to the clasp 20 by passing the male portion through an appropriate fastener hole 15, folding the clasp 20 onto itself, and securing the male to the female portion of the clasp 20. Once attached or sized, an excess length of the strap 14 may be removed by cutting and severing the excess portion from the strap 14.

After some duration of use, the strap 14 and clasp 20 may be destructively detached from the main body 12, such as by cutting it with scissors in order to remove the medical band 11 from the patient. It is contemplated that the main body 12 may be reattached to the patient using another re-attachment strap (not illustrated) using the slots 22 and 24, For example, the new strap may be inserted through the first slot 22, starting on the front side of the main body 12, then passing the strap along the back side of the main body 12, and then out the second slot 24, such that the strap 14 passes from the back of the main body 12 to the front of the main body 12. The new strap may be made into a loop again using a second clasp on the new strap. This may be useful if the nurse or patient accidently removes the medical band 11, or if the medical band 11 is too small for the patient's wrist.

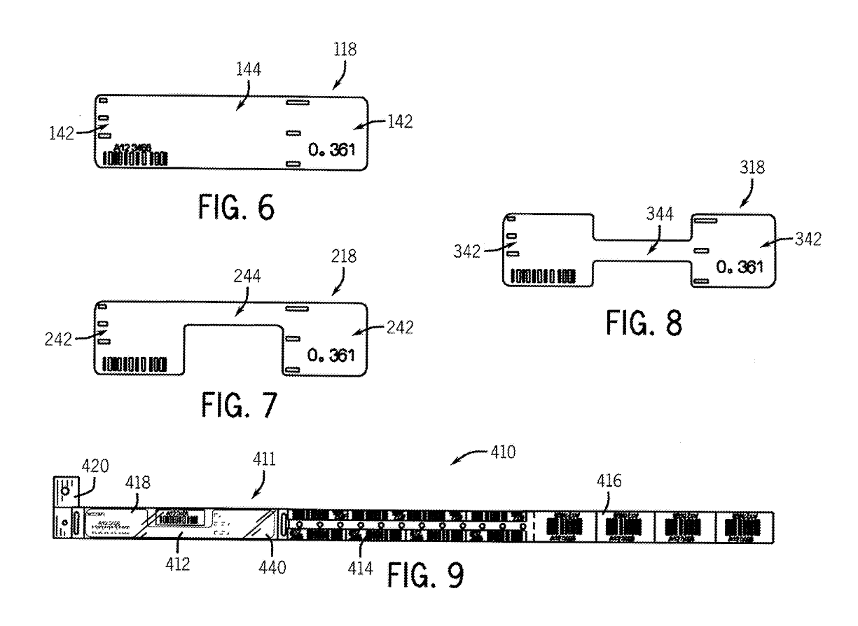

It will be appreciated that the illustrated flag label 18 in FIGS. 1 through 4 is just one example of a flag label. FIGS. 6 through 8 illustrate additional flag label geometries that are contemplated as being within the scope of this disclosure.

For example, in FIG. 6, according to a first alternative embodiment of the flag label 118, the intermediate support bridge 144 extends between the flag sections 142 and between the lateral edges of the flag label 118, such that the flag label 118 is a rectangular shape, albeit with rounded corners.

In a second alternative embodiment of the flag label 218 shown in FIG. 7, the intermediate support bridge 244 extends along the opposite lateral edge of the flag label 218 between the flag sections 242 in comparison to the embodiment illustrated in FIGS. 1-4, such that it forms an inverted U shape. The dimensions of the second alternative embodiment of the flag label 218 may be similar to the dimensions of the flag label 18 illustrated in FIGS. 1-4.

As shown in FIG. 7, in a third alternative embodiment of the flag label 318, the intermediate support bridge 344 extends between the flag sections 342 at a location between the top and bottom of the flag label 318 forming a barbell-shaped peripheral edge. More specifically, the intermediate support bridge 244 extends along the halfway distance between the top and bottom of the flag label 318 to form a sideways I shape that is symmetrical (sans printed data on the top surface) along two axes including the width and length directions.

Finally, with reference to FIG. 9, an alternative form of the medical band assembly 410 is illustrated to stress that the medical band assembly 410 may also come in a variety of shapes and sizes. For example, in the embodiment shown in FIG. 1, the medical band assembly 10 may have a wider main body 12 and flag label 18. By providing a wider main body 12 and flag label 18, more information may be provided for visual inspection. Whereas, in the medical band 410, the main body 412 and strap 414 of the medical band 411, as well as the detachable tail 416 may have a consistent or uniform width over its length (except for the clasp 420). Even with such dimension changes, the flag label 418 may still be initially supported under the transparent laminate shield 440 and detachable from the main body 412 as described above.

As noted above, it should be appreciated that various other modifications and variations to the preferred embodiments can be made within the spirit and scope of the invention. Therefore, the invention should not be limited to the described embodiments. To ascertain the full scope of the invention, the following claims should be referenced.

1. A medical band assembly comprising:
a medical band having an elongated body with opposing surfaces bounded at their edges by a periphery, the medical band extending from a main body to a strap in which the strap is attachable to the main body to form a loop from the medical band;
a flag label supported on one of the opposing surfaces of the main body of the medical band and being separable from the medical band for attachment to an object to be labeled, the flag label including a pair of flag sections joined to one another by an intermediate support bridge;
wherein, when the flag label is separated from the medical band, the pair of flag sections are foldable onto one another to join a contacted pair of their surfaces together such that the intermediate support bridge is wrapable around the object to be labeled by the flag label.

2. The medical band assembly of claim 1, wherein the object is cylindrical in shape, and wherein the intermediate support bridge wraps around the circumference of the object.

3. The medical band assembly of claim 2, wherein the object is a test tube.

4. The medical band assembly of claim 1, further comprising a transparent laminate shield attached to the main body of the medical band in which the transparent laminate shield is liftable from the main body to separate at least a portion of the transparent laminate shield from the main body.

5. The medical band assembly of claim 4, wherein the flag label is disposed between the medical band and the transparent laminate shield.

6. The medical band assembly of claim 5, wherein the transparent laminate shield has an adhesive backing.

7. The medical band assembly of claim 1, wherein each flag section has a full lateral length between 3 and 6 inches and the intermediate support bridge has a lateral length of 1.6 inches and a width between 0.2 and 1 inches.

8. The medical band assembly of claim 1, further comprising a detachable tail having a plurality of tail labels supported thereon and wherein the medical band has at least one main label supported thereon.

9. The medical band assembly of claim 8, wherein the flag label, the plurality of the tail labels, and at least one the main label include a computer readable representation of data, and
wherein the computer readable representation of data is the same for the flag label, the plurality of tail labels, and at least one main label.

10. The medical band assembly of claim 9, wherein the flag label further includes a human readable representation of data.

11. A method of attaching a flag label to an object, the method comprising:
detaching the flag label from a medical band assembly, in which the medical band assembly comprises:
a medical band having a main body attached to a strap in which the strap is attachable to the main body to form a loop from the medical band;
the flag label having a first and second flag sections separated by an intermediate support bridge; and
an adhesive applied to the back of at least one of the first and second flag sections
wrapping at least a portion of the intermediate support bridge around the object;
adhering the first flag section to the second flag section thereby attaching the flag label around the object.

12. The method of claim 11, further comprising:
aligning the first flag section with the second flag section prior to adhering the first flag section with the second flag section.

13. The method of claim 11, further comprising:
attaching the medical band to a wrist of a patient by attaching the strap to the main body to form a loop around the wrist.

14. The method of claim 11, wherein the flag label is initially releasably adhered to the main body of the medical band prior to the detaching step.

15. The method of claim 11, wherein the medical band further includes a transparent laminate shield having an adhesive backing protected by a releasable liner.

16. The method of claim 15, further comprising:
removing the releasable liner from the transparent laminate shield and adhering the transparent laminate shield to the medical band.

17. The method of claim 11, wherein the adhesive is supported on at least a portion of the intermediate support bridge and wherein the method further comprises the step of adhering at least a portion of the intermediate support bridge to the object.

18. The method of claim 11, wherein the flag sections have a computer readable representation of data.

19. The method of claim 11, wherein the intermediate support bridge is narrower in a width direction than the flag sections.
